(19) United States
(12) Patent Application Publication
(10) Pub. No.: US 2018/0174691 A1
(43) Pub. Date: Jun. 21, 2018

(54) SYSTEM AND METHOD FOR FACILITATING VISUALIZATION OF INTERACTIONS IN A NETWORK OF CARE PROVIDERS

(71) Applicant: Koninklijke Philips N.V., Eindhoven (NL)

(72) Inventors: Sharon Williams, Eindhoven (NL); Ilona Owusu, Eindhoven (NL); Vincentius Paulus Buil, Eindhoven (NL); Payaal Patel, Reading, MA (US)

(21) Appl. No.: 15/848,123

(22) Filed: Dec. 20, 2017

Related U.S. Application Data
(60) Provisional application No. 62/437,079, filed on Dec. 21, 2016.

(51) Int. Cl. G16H 80/00; G16H 10/60; G16H 40/20; G06F 3/048; G06T 11/20

(52) U.S. Cl. CPC G16H 80/00; G16H 10/60; G06T 11/206; G06F 3/048; G16H 40/20

(57) ABSTRACT

This disclosure describes systems and methods for facilitating visualization of interactions in a network of care providers. The system may determine a first care provider type associated with a first care provider; receive information related to one or more interactions of one or more subjects with one or more other care providers; determine at least a portion of the interaction-related information relevant to the first care provider; and effectuate presentation of the interaction-related information portion such that the presentation of the interaction-related information portion reflects one or more aspects of the one or more interactions of a first subject not originating from the first care provider.

### CROSS-REFERENCE TO RELATED APPLICATIONS

The present application claims priority to and benefit of U.S. Provisional Application No. 62/437,079, filed Dec. 21, 2016, its entirety of which is hereby incorporated by reference herein.

### BACKGROUND OF THE INVENTION

### 1. Field

The present disclosure pertains to a system and method for facilitating visualization of interactions in a network of care providers.

### 2. Description of the Related Art

The increasing adoption of electronic medical records has made information more accessible to care providers. However, increased access to information may not necessarily translate into use of the information. A patient's electronic medical record may contain a large amount of unstructured textual information. With patient medical records becoming increasing dense due to an aging population and growing multimorbidity, it may be difficult to access, organize, and navigate patient medical records in a way that facilitates a care provider's ability to understand the information. A comprehensive review of a patient's medical record may require the care provider to examine multiple documents while mentally noting issues related to the current clinical context and filtering out unrelated information. Moreover, in some scenarios, the relevancy of certain information presented to care providers and/or other users via conventional systems may not be easily ascertainable.

As an example of the foregoing, conventional systems may provide care providers and/or other users with user interfaces that are overcrowded with information corresponding to one or more patients or lack certain relevant information (e.g., as a result of attempting to avoid user interface overcrowding). Thus, even with the advent of electronic medical records and computer-assisted information sharing systems, a care provider may not identify, or readily identify relevant information with respect to a patient such as those related to interactions with other care providers (or other information), for example, in scenarios where there is a plethora of information related to the patient (although not necessarily relevant for an appointment with the care provider), and where those interactions did not originate from the care provider.

### SUMMARY OF THE INVENTION

Accordingly, one or more aspects of the present disclosure relate to a system configured to facilitate, improve and enable visualization of interactions in a network of care providers.

The system herein described comprises one or more processors and/or other components. The one or more processors are configured by machine-readable instructions to determine a first care provider type associated with a first care provider. The one or more processors are configured by machine-readable instructions to receive information related to one or more interactions of one or more patients, for instance subjects, individuals, users or otherwise person with other care providers. The interaction-related information includes information related to one or more interactions of a first patient with at least one of the other care providers. The one or more interactions of the first patient may be not originating from the first care provider. The one or more processors are configured by machine-readable instructions to determine at least a portion of the interaction-related information relevant to the first care provider from the one or more interactions. The interaction-related information portion is determined to be relevant to the first care provider (i) based on the one or more interactions of the first patient being relevant to the first care provider type and (ii) despite the one or more interactions of the first patient not originating from the first care provider. The one or more processors are configured by machine-readable instructions to effectuate presentation, on a user interface, for instance a graphical user interface (GUI), of the interaction-related information portion such that the presentation of the interaction-related information portion reflects one or more aspects of the one or more interactions of the first patient not originating from the first care provider.

In some embodiments, system may obtain information related to one or more interactions of a patient with other care providers, and present at least a portion of the interaction-related information to a first care provider, for example, even if the interactions of the patient did not originate from the first care provider. As an example, system 10 may determine a care provider type of the first care provider and determine that the interaction-related information portion is relevant to the first care provider based on the interactions of the patient being relevant to the care provider type of the first care provider. Responsive to such determination, system may present the interaction-related information portion to the first care provider. As such, in addition to addressing the foregoing technical issues, system may reduce communication gaps between patients and care providers along with one or more undesirable consequences resulting therefrom. These consequences may include less efficient consultations, wrong treatments being prescribed, poor adherence to medication, and/or other consequences. As an example, by identifying relevant interaction-related information for a care provider (e.g., even when the related interactions did not originate from the care provider), the care provider can utilize the identified interaction-related information to avoid such undesirable consequences. As another example, tracking the intensity of interactions between patients and care providers and generating one or more visualizations of such information may facilitate the ease with which the care provider may identify relevant information, which, for example, may in turn facilitate a more efficient conversation between the care providers and patients (and their formal/informal care providers). Additionally, or alternatively, tracking and visualizing the intensity of interactions between patients and care providers may facilitate care providers to prescribe better treatments and/or better understand issues with medication compliance.

Another aspect of the present disclosure relates to a method for facilitating visualization of interactions in a network of care providers with a system, which system comprises one or more processors and/or other components. The method comprises determining a first care provider type associated with a first care provider. The method comprises receiving information related to one or more interactions of one or more patients, for instance subject, individuals, users or otherwise person, with other care providers. The interaction-related information includes information related to one or more interactions of a first patient with at least one of the other care providers. The one or more interactions of the first patient may be not originating from the first care provider. The method comprises determining at least a portion of the interaction-related information relevant to the first care provider from the one or more interactions. The interaction-related information portion is determined to be relevant to the first care provider (i) based on the one or more interactions of the first patient being relevant to the first care provider type and (ii) despite the one or more interactions of the first patient not originating from the first care provider. The method comprises effectuating presentation of the interaction-related information portion, on a user interface, for instance a graphical user interface (GUI), such that the presentation of the interaction-related information portion reflects one or more aspects of the one or more interactions of the first patient not originating from the first care provider.

The method according to the present disclosure comprises analogous advantage as the system configured for facilitating, improving and enabling visualization of interactions in a network of care providers herein described.

Still another aspect of present disclosure relates to a system for facilitating visualization of interactions in a network of care providers. The system comprises means for determining a first care provider type associated with a first care provider. The system comprises means for receiving information related to one or more interactions of one or more patients, for instance subject, individuals, users or otherwise person, with other care providers. The interaction-related information includes information related to one or more interactions of a first patient with at least one of the other care providers. The one or more interactions of the first patient may be not originating from the first care provider. The system comprises means for determining at least a portion of the interaction-related information relevant to the first care provider from the one or more interactions. The interaction-related information portion is determined to be relevant to the first care provider (i) based on the one or more interactions of the first patient being relevant to the first care provider type and (ii) despite the one or more interactions of the first patient not originating from the first care provider. The system comprises means for effectuating presentation of the interaction-related information portion such that the presentation, on a user interface, for instance a graphical user interface (GUI), of the interaction-related information portion reflects one or more aspects of the one or more interactions of the first patient not originating from the first care provider.

These and other aspects of the invention are apparent from and will be elucidated with reference to the embodiments described hereinafter.

It will be appreciated by those skilled in the art that two or more of the above-mentioned options, implementations, and/or aspects of the invention may be combined in any way deemed useful.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

These and other aspects of the system and the method according to the invention will be further elucidated and described with reference to the drawing, in which:

FIG. 1 is a schematic illustration of a system configured to facilitate visualization of interactions in a network of care providers according to one or more embodiments.

FIG. 2 illustrates an interactive visualization personalized for a care provider according to one or more embodiments.

FIG. 3 illustrates a linear visualization of interaction intensity of patient and care providers over time according to one or more embodiments.

FIG. 4 illustrates patient interactions with care providers over time with related information streams between care providers according to one or more embodiments.

FIG. 5 illustrates searching and filtering interactions between patient and care providers according to one or more embodiments.

FIG. 6 illustrates a method for facilitating visualization of interactions in a network of care providers according to one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

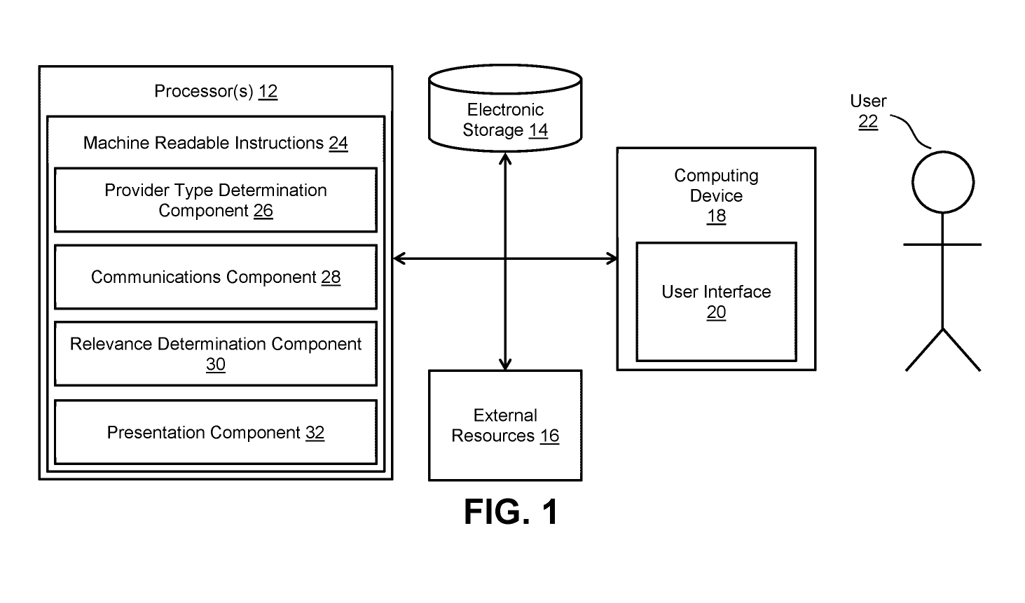

FIG. 1 is a schematic illustration of a system 10 configured to facilitate visualization of interactions in a network of care providers. System 10 is configured to effectuate presentation of a dynamic and interactive visualization of interactions between patients and care providers.

In some embodiments, system 10 provides a user interface that facilitates a dynamic and interactive visualization of a patient's interactions with care providers, for example, to provide a more user-friendly user interface via which an individual (e.g., a care provider or other user) can more easily identify relevant interactions and other information with respect to the patient. As an example, a care provider (or other user) may be presented with the patient's interactions organized into interactions with individual healthcare entities via the user interface. System 10 may present, via the user interface, the patient's interactions with individual care providers at an individual healthcare entity responsive to a user selection of an individual healthcare entity. Additionally, or alternatively system 10 may visualize, via the user interface, an intensity of the patient's interactions with individual care providers by varying a thickness of an outline (e.g., a border) encompassing each of the individual care providers (represented on the user interface).

In some embodiments, system 10 comprises one or more of a processor 12, electronic storage 14, external resources 16, a computing device 18, and/or other components. Processor 12 is configured to provide information processing capabilities in system 10. As such, processor 12 may comprise one or more of a digital processor, an analog processor, a digital circuit designed to process information, an analog circuit designed to process information, a state machine, and/or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., a server, computing device 18 associated with user 22, devices that are part of external resources 16, and/or other devices.)

As shown in FIG. 1, processor 12 is configured via machine-readable instructions 24 to execute one or more computer program components. The one or more computer program components may comprise one or more of a provider type determination component 26, a communications component 28, a relevance determination component 30, a presentation component 32, and/or other components. Processor 12 may be configured to execute components 26, 28, 30, and/or 32 by software; hardware; firmware; some combination of software, hardware, and/or firmware; and/or other mechanisms for configuring processing capabilities on processor 12.

It should be appreciated that although components 26, 28, 30, and 32 are illustrated in FIG. 1 as being co-located within a single processing unit, in embodiments in which processor 12 comprises multiple processing units, one or more of components 26, 28, 30, and/or 32 may be located remotely from the other components. The description of the functionality provided by the different components 26, 28, 30, and/or 32 described below is for illustrative purposes, and is not intended to be limiting, as any of components 26, 28, 30, and/or 32 may provide more or less functionality than is described. For example, one or more of components 26, 28, 30, and/or 32 may be eliminated, and some or all of its functionality may be provided by other components 26, 28, 30, and/or 32. As another example, processor 12 may be configured to execute one or more additional components that may perform some or all of the functionality attributed below to one of components 26, 28, 30, and/or 32.

Provider type determination component 26 is configured to determine a first care provider type associated with a first care provider. In some embodiments, the first care provider includes formal care providers (e.g., cardiologist, diabetic specialist nurse, health coach, home nurse, or other care provider), informal care providers (e.g., family member), and/or other care providers. In some embodiments, provider type determination component 26 is configured to determine one or more parameters relevant to the first care provider type. The one or more relevant parameters include one or more keywords, conditions, illnesses, diagnosis, medication, or interaction types relevant to the first care provider type. In some embodiments, the one or more relevant parameters are obtained manually from the first care provider. In some embodiments, the one or more relevant parameters are obtained automatically. For example, the one or more relevant parameters may be obtained via a learning system (e.g., neural networks, deep learning). By way of a non-limiting example, Table 1 illustrates possible parameters relevant to a cardiologist and a diabetic specialist nurse. As shown in Table 1, certain symptoms, diseases and conditions are relevant for both the cardiologist and the diabetic specialist nurse. As such, patient interactions with the two care providers related to the common symptoms, diseases and conditions may be visualized for both care providers.

TABLE 1

| | Cardiologist | Diabetic specialist nurse |
|---|---|---|
| Symptoms | Chest pain, short breath, tired, | Tired, headaches, dizziness, flaws, |
| | headaches, dizziness, flaws, | varicose veins, wounds, peeing |
| | varicose veins, peeing often | often, blurred vision |
| Diseases/ | CHF, CVD, CAD, high cholesterol, | Diabetes Type 1 and 2, vascular |
| conditions | high BP | conditions, heart conditions |
| Medication | ACE Inhibitors, ARBs, Beta- | Insulin, Alpha-glucosidase |
| | Blockers, Diuretics | inhibitors, Biguanides |

As illustrated in Table 1, several symptoms, diseases and conditions may only be relevant to a particular care provider and may not overlap with parameters relevant to another care provider. For example, symptoms including chest pain and short breath are may be only relevant to the cardiologist. Therefore, patient interactions related to chest pain and short breath may only be visualized for the cardiologist.

Returning to FIG. 1, communications component 28 is configured to receive information related to one or more interactions of one or more patients with other care providers. In some embodiments, the interaction-related information includes information related to one or more interactions of a first patient with at least one of the other care providers. In some embodiments, the one or more interactions of the first patient may not be originating from the first care provider. In some embodiments, the one or more interactions of the first patient may include interactions with the first care provider.

In some embodiments, the received information is related to appointment data corresponding to the first patient. The appointment data includes a time of interaction, a place of interaction, care provider type, a frequency of interactions, an intensity of interaction, and/or other information. In some embodiments, the received information is related to interaction content. The interaction content includes discussed diagnosis, treatment steps, lab orders, forwarding to the other care providers, medication subscriptions, self-reported compliance to therapy, experienced symptoms and side effects, and/or other information.

Relevance determination component 30 is configured to determine at least a portion of the interaction-related information relevant to the first care provider. In some embodiments, relevance determination component 30 determines the interaction-related information portion to be relevant to the first care provider based on the one or more interactions of the first patient being relevant to the first care provider type. In some embodiments, relevance determination component 30 is configured to determine the interaction-related information relevant to the first care provider despite the one or more interactions of the first patient not originating from the first care provider. For example, an appointment with at least one of the other care providers may be determined to be relevant to the first care provider even though the care provider didn't refer the patient to the other care provider. As another example, a lab test requested by at least one of the other care providers may be determined to be relevant to the first care provider even though the first care provider is unaware of the lab test request (e.g., and, thus, had not yet requested the lab test results).

In some embodiments, relevance determination component 30 is configured to filter the received information for the first patient during a time window. For example, relevance determination component 30 may be configured to pass only direct interactions of the first patient with care providers. In some embodiments, relevance determination component 30 may be configured to pass interactions between care providers discussing one or more health aspects of the first patient. In some embodiments, relevance determination component 30 may be configured to pass information related to the one or more interactions of the first patient that took place in the past week, month, quarter, year or any other time period. In some embodiments, one or more settings related to filtering information may be adjusted by the first care provider and/or other users.

In some embodiments, relevance determination component 30 is configured to determine the interaction-related information portion as being relevant to the first care provider based on the one or more interactions of the first patient matching the one or more parameters relevant to the first care provider type. In some embodiments, relevance determination component 30 determines the interaction-related information portion as being relevant to the first care provider despite the one or more interactions of the first patient not originating from the first care provider. For example, a visit to a dentist and an optometrist/ophthalmologist following an intervention by a neurologist may be relevant to the neurologist as the interactions with the dentist and the optometrist/ophthalmologist may interfere with the treatment prescribed by the neurologist.

In some embodiments, relevance determination component 30 is configured to classify the one or more interactions of the first patient with the other care providers based on the one or more interactions of the first patient matching the one or more parameters relevant to the first care provider type. For example, relevance determination component 30 may classify acute events, emergencies, hospital admission, and/or other interactions as very important. As another example, relevance determination component 30 may classify new and/or altered diagnosis, new and/or altered treatment, new and/or altered medication, and/or other interactions as important. In yet another example, relevance determination component 30 may classify instructions to the first patient, regular checkup, and/or other interactions as less important. In yet another example, relevance determination component 30 may classify changes with respect to specific (diagnosed) diseases, conditions, symptoms, medication, treatments, and/or other medical information (and/or the first patient's experiences with the changes) as very relevant, while classifying changes with respect to other diseases, conditions, symptoms, medication, and/or treatments as less relevant. In some embodiments, the classification may be based the one or more parameters relevant to the first care provider type.

In some embodiments, relevance determination component 30 is configured to identify missing appointments and/or interactions that should have taken place. For example, care providers may request patients, informal care providers, and/or other care providers to make follow-up appointments. Such requests may not always be followed up, and/or alternatively planned appointments may not take place or may be rescheduled. Relevance determination component 30 is configured to identify appointment requests in the interaction content. In some embodiments, relevance determination component 30 determines if the appointment requests have resulted in appointments in the appointment data within a predetermined time period. In some embodiments, relevance determination component 30 identifies due appointments in the appointment data and determines whether an appointment did take place by checking for corresponding entries in the interaction content. In some embodiments, relevance determination component 30 is configured to check for appointments in the appointment data that are rescheduled in which the time between original date and new date is more than a predetermined time period.

In some embodiments, relevance determination component 30 is configured to notify the first care provider of a failed appointment. Relevance determination component 30 is configured to identify whether the failed appointment is due to an appointment not being scheduled in time, a scheduled appointment not taking place, the appointment being rescheduled, or other criteria. In some embodiments, relevance determination component 30 is configured to determine whether appointment topic content of the failed appointment is relevant to the first care provider based on the appointment topic content matching the one or more parameters relevant to the first care provider type. Responsive to a determination that the appointment topic content relevance exceeds a predetermined threshold, relevance determination component 30 is configured to notify the first care provider with regard to the failed appointment (e.g. via in-system message, e-mail, SMS, mobile application message, etc.). For example, a missed appointment related to the first patient experiencing severe chest pain, shortness of breath, and sweating may exceed the predetermined threshold of relevance for a cardiologist; therefore, the cardiologist may be notified of the missed appointment. As another example, a missed appointment related to the first patient experiencing slight body aches, a mild headache, low-grade fever, sore throat, and/or other symptoms may not exceed the predetermined threshold of relevance for a cardiologist; therefore, the cardiologist may not be notified of the missed appointment.

Presentation component 32 is configured to effectuate presentation of the interaction-related information portion such that the presentation of the interaction-related information portion reflects one or more aspects of the one or more interactions of the first patient not originating from the first care provider. In some embodiments, presentation component 32 is configured to effectuate presentation of one or more healthcare entities (e.g., hospitals, pharmacy, or other healthcare entities). In some embodiments, presentation component 32 is configured to distinguish between individual frequencies of interactions between the first patient with the one or more healthcare entities by varying one or more of a thickness, length, or color of one or more lines between the first patient and the one or more healthcare entities. In some embodiments, presentation component 32 is configured to highlight the most recent interaction between the first patient and the one or more healthcare entities. In some embodiments, presentation component 32 is configured to effectuate presentation of the first patient's interaction with the other care providers at an individual one of the one or more healthcare entities responsive to a selection of the one or more healthcare entities.

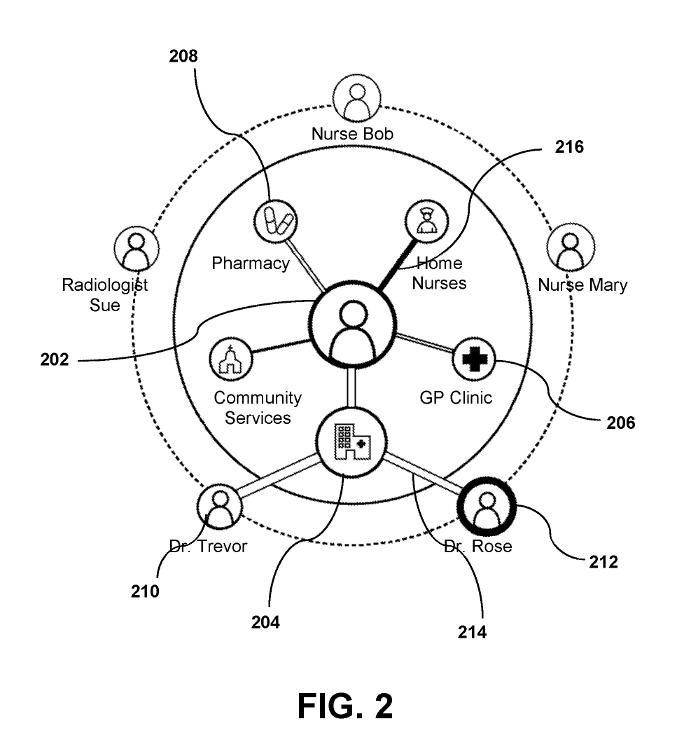

By way of a non-limiting example, FIG. 2 illustrates an interactive visualization personalized for a care provider according to one or more embodiments. As shown in FIG. 2, first patient 202 is visualized in a center of an interactive map corresponding to first patient 202's care network and to first patient 202's interactions with the care network. In some embodiments, Hospital 204, GP clinic 206, pharmacy 208, and/or other healthcare entities are visualized in a first circle around first patient 202. The thickness of lines 214, 216, and/or other lines extending between first patient 202 and Hospital 204, GP clinic 206, pharmacy 208, and/or other healthcare entities may indicate a frequency of interaction (e.g. one or more interactions per week=thick, one to four interactions per month=medium, one to three interactions per a year=thin). In some embodiments, presentation component 32 may highlight lines representing the most recent interaction between first patient 202 and one or more of Hospital 204, GP clinic 206, pharmacy 208, or other healthcare entities (e.g., using orange colored lines). In some embodiments, responsive to the first care provider and/or other users selecting Hospital 204, GP clinic 206, pharmacy 208, or other healthcare entities, a second circle may be presented. The second circle may include care providers (e.g., Dr. Trevor 210, Dr. Rose 212, and/or other care providers) who interacted with first patient 202 at an individual one of Hospital 204, GP clinic 206, pharmacy 208, or the other healthcare entities. In some embodiments, presentation component 32, responsive to a selection, by the first care provider and/or other users of a line between first patient 202 and an individual one of Hospital 204, GP clinic 206, pharmacy 208, or the other healthcare entities, presents dates of interactions between first patient 202 and the individual one of Hospital 204, GP clinic 206, pharmacy 208, or the other healthcare entities. In some embodiments, responsive to a selection, by the first care provider and/or other users, of the presented dates of interaction, presentation component 32 is configured to effectuate presentation of details corresponding to the selected interaction (e.g., appointment data, topics discussed).

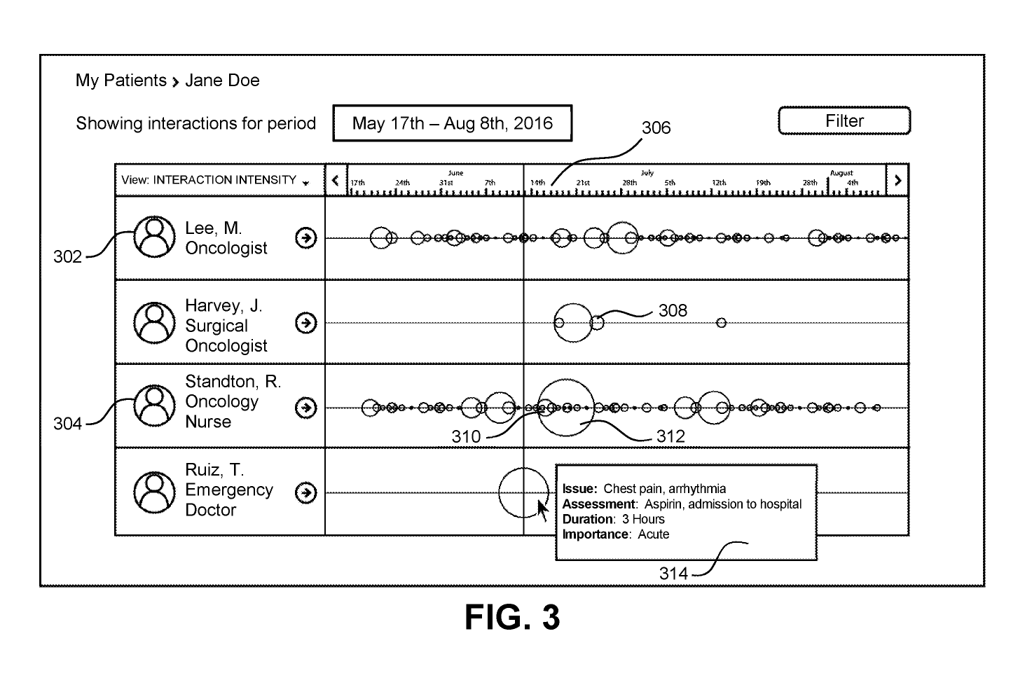

FIG. 3 illustrates a linear visualization of interaction intensity of patient and care providers over time according to one or more embodiments. As shown in FIG. 3, a first patient's interactions with each care provider have been visualized in a linear graph. For example, first patient's interactions with oncologist 302 have been visualized on a first line and first patient's interactions with oncology nurse 304 have been visualized on a third line. As depicted in FIG. 3, first patient's interactions with multiple care providers have been visualized during a time window 306. Each circle (e.g., 308, 310, 312, and/or other circles) shown in FIG. 3 represents an individual interaction with the care providers (e.g., oncologist 302, oncology nurse 304, and/or other care providers). In some embodiments, a size of each circle illustrates an intensity of interaction between first patient and the corresponding care provider. In some embodiments, one or more circles may be overlapping (e.g., 310, 312) due to the linear time mapping of interactions. In some embodiments, responsive to a selection, by the first care provider and/or other users of an individual interaction circle, presentation component 32 is configured to effectuate presentation of details 314 corresponding to the selected interaction.

Returning to FIG. 1, presentation component 32 is configured to effectuate presentation of the other care providers. In some embodiments, presentation component 32 is configured to sort the presentation of the other care providers based on a chronological order of the first patient's one or more interactions with the other care providers. In some embodiments, presentation component 32 effectuates presentation of one or more interactions between the other care providers. In some embodiments, presentation component 32 is configured to distinguish between intensities of interactions between the first patient and the other care providers by varying one or more of a thickness, a size, or a color of an outline corresponding to individual ones of the other care providers.

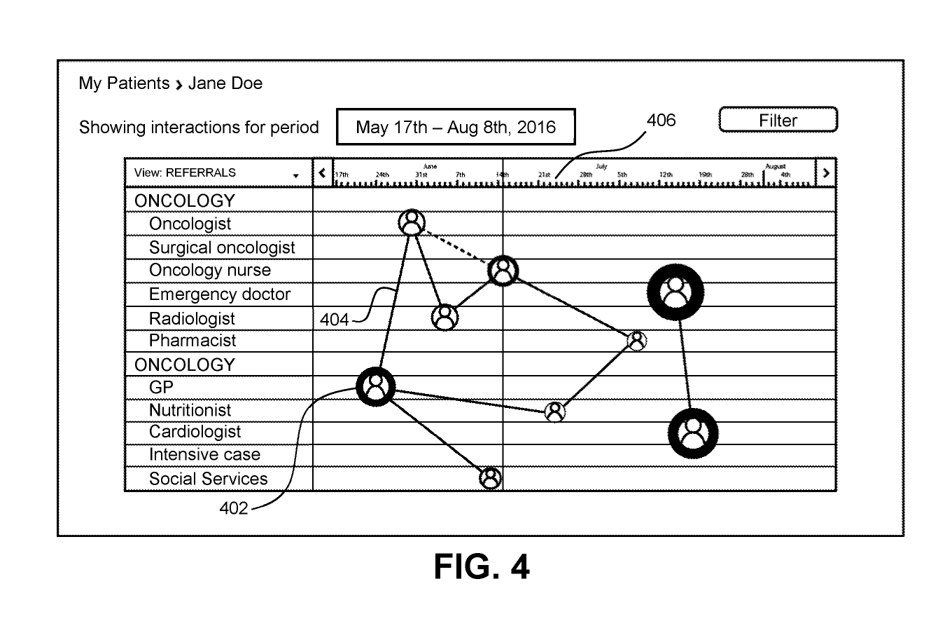

By way of a non-limiting example, FIG. 4 illustrates patient interactions with care providers over time with related information streams between care providers according to one or more embodiments. In FIG. 4, care providers conforming to the first patient's network of care providers have been visualized. As shown in FIG. 4, interactions (e.g., 404) between individual ones of the other care providers (e.g. care provider 402) has been have been visualized during a time window 406. In some embodiments, presentation component 32 is configured to vary an outline corresponding to individual ones of the other care providers to illustrate an intensity of interaction of a given care provider of the other care providers with the first patient.

Returning to FIG. 1, presentation component 32 is configured to provide an overview to the first patient of all of the care providers in the first patient's network of care providers. In some embodiments, the overview may be navigated. In some embodiments, presentation component 32 facilitates searching and/or filtering contents of the overview by historically visualizing the content through a timeline, grouping by specialty, primary or secondary care, distance or location, insurance coverage, expense, and/or other visualizations.

In some embodiments, presentation component 32 is configured to provide an overview to the first care provider and/or the other care providers of all of the care providers in the first patient's network of care providers. In some embodiments, each care provider may be connected to a discipline related to the first patient's electronic medical record (EMR). In some embodiments, presentation component 32 is configured such that the provided overview may be navigated historically through a timeline by giving an overview of the first patient's current versus past status.

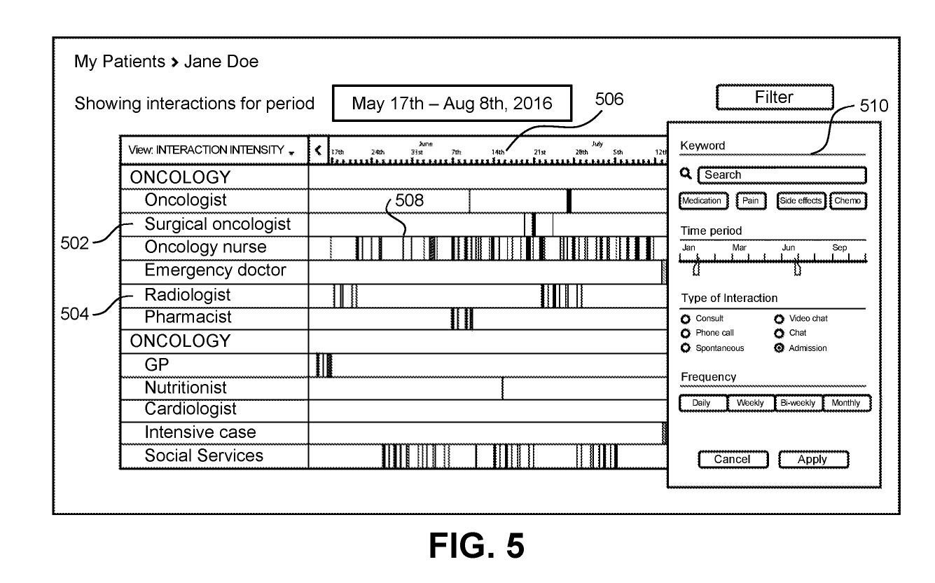

By way of a non-limiting example, FIG. 5 illustrates searching and filtering interactions between patient and care providers according to one or more embodiments. As shown in FIG. 5, first patient's interactions (e.g., 508) with the other care providers (e.g., 502, 504) have been visualized in a linear time window 506. In FIG. 5, the first care provider, the other care providers, and/or other users may search and filter interactions using interface 510. For example, presentation component 32 may effectuate presentation of all of the interactions using blue bars, interactions fully matching with the search and filter criteria with red bars, and interactions partially matching with the search and filter criteria with orange bars. In some embodiments, a thickness of the bars may describe the length of interaction, and the color of the bars may represent an importance of the interaction (e.g., consultation=blue, hospital admission=orange, emergency admission=red). In some embodiments, presentation component 32 may facilitate the first care provider and/or other users interacting with a first patient having complicated clinical pathways due to comorbidities and/or iterative care plans (e.g., a cancer patient with chronic heart failure) to find relevant interactions and corresponding intensities according to the one or more parameters relevant to the first care provider type. For example, presentation component 32 may facilitate a filter function based on metadata and/or a free form search based on tags or keywords related to the appointment data and interaction content corresponding to the first patient.

In some embodiments, presentation component 32 is configured to automatically adjust the visualization based on the user interaction behavior while safeguarding completeness. For example, users may (structurally) select certain information according to their preferences while interacting with the interactive visualization (e.g., the first care provider may structurally inspect interactions with the pharmacy to see developments in the first patient's medication). Responsive to determining user's interaction behavior, presentation component 32 may show the preferred information immediately each time the user opens the interactive visualization.

In some embodiments, the user (e.g., the first care provider) may manually deselect certain information because the user may find the information distracting. In some embodiments, responsive to a determination that deselecting the information would not risk the user missing important information, presentation component 32 may automate such deselection. In some embodiments, information relevance component 30 may determine whether deselecting the information would risk the user missing important based on the deselected information matching the one or more parameters relevant to the first care provider type. For example if the user prefers that more recent interactions are not highlighted in the visualization, information relevance component 30 may determine that switching highlighting off would risk the user missing important information. In some embodiments, presentation component 32 may switch highlighting off and may switch highlighting on again responsive to an occurrence of an important interaction.

Electronic storage 14 comprises electronic storage media that electronically stores information. The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, processor 12, etc.). In some embodiments, electronic storage 14 may be located in a server together with processor 12, in a server that is part of external resources 16, in computing device 18 associated with user 22, and/or other users, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processor 12, information received via computing device 18 and/or other external computing systems, information received from external resources 16, and/or other information that enables system 10 to function as described herein.

External resources 16 include sources of information (e.g., databases, websites, etc.), external entities participating with system 10 (e.g., a Patient Administration System at a hospital), one or more servers outside of system 10, a network (e.g., the internet), electronic storage, equipment related to Wi-Fi technology, equipment related to Bluetooth® technology, data entry devices, computing devices associated with individual users, and/or other resources. In some implementations, some or all of the functionality attributed herein to external resources 16 may be provided by resources included in system 10. External resources 16 may be configured to communicate with processor 12, computing device 18, electronic storage 14, and/or other components of system 10 via wired and/or wireless connections, via a network (e.g., a local area network and/or the internet), via cellular technology, via Wi-Fi technology, and/or via other resources.

In some embodiments, external resources 16 include appointment data and interaction content provided on a Patient Administration System (PAS) in a hospital. For example, the PAS may detail all patient contact with the hospital, both outpatient and inpatient, and may contain appointment related data with formal care providers.

In some embodiments, external resources 16 include patient portals and patient applications (e.g., Philips CarePartners Mobile app). Patient portals and patient applications may include appointment information and a log of electronic contact the first patient has had with care providers. The electronic appointment data, as well as chat and video-call activities through apps or portals provide information on when, for how long and how often the first patient has had contact with care providers.

In some embodiments, external resources 16 include information related to a speaker identified during an interaction recording, information related to a type of interaction (e.g., instructional, descriptive/informational, topic), information related to a stress level of the first patient during the interaction (e.g., electrodermal activity sensed via skin resistance or skin conductance, heart rate and/or breathing rate response and/or variability sensed via optical, galvanic, and/or other types of sensors, facial expressions sensed via camera based facial tracking technologies, muscle tension sensors and/or other sensors, voice prosody sensed via voice tone analysis technologies, and/or any other technology, system, or method to detect the first patient's emotions), information related to location matching of the first patient, the first care provider, and/or the other care providers that captures non-scheduled interactions (e.g., via GPS, WiFi ranging, Bluetooth proximity of smart devices worn by the first patient, the first care provider, and/or the other care providers, handshake detection via body coupled communication within the smart devices, first patient and/or care provider identification via cameras in hospitals), and/or other information.

In some embodiments, the contents of interactions between the first patient and the first care provider and/or the other care providers (e.g. diagnosis, treatment, results) are stored in the patient's medical record (e.g., Electronic Medical Record, Personal Health Record). In some embodiments, the interaction content may include an audio recording of the conversation, audio/video conversation transcription via dictation software, whether the interaction has taken place live or via a telephone or video call, a list of discussed subjects determined by topic analysis of the recorded conversation (e.g., word spotting in the transcription or via speech recognition, and/or other information.

Computing device 18 is configured to provide an interface between user 22, and/or other users and system 10. Computing device 18 is configured to provide information to and/or receive information from the user 22, and/or other users. For example, computing device 18 is configured to present a user interface 20 to user 22 (e.g., a first care provider) to facilitate presentation of a first patient's interactions with other care providers. In some embodiments, user interface 20 includes a plurality of separate interfaces associated with computing device 18, processor(s) 12 and/or other components of system 10.

In some embodiments, computing device 18 is configured to provide user interface 20, processing capabilities, databases, and/or electronic storage to system 10. As such, computing device 18 may include processor(s) 12, electronic storage 14, external resources 16, and/or other components of system 10. In some embodiments, computing device 18 is connected to a network (e.g., the internet). In some embodiments, computing device 18 does not include processor(s) 12, electronic storage 14, external resources 16, and/or other components of system 10, but instead communicate with these components via the network. The connection to the network may be wireless or wired. For example, processor(s) 12 may be located in a remote server and may wirelessly cause display of user interface 20 to user 22 on computing device 18. In some embodiments, computing device 18 is a laptop, a personal computer, a smartphone, a tablet computer, a smart watch, an activity tracker, and/or other computing devices. Examples of user input devices suitable for inclusion in computing device 18 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that computing device 18 includes a removable storage interface. In this example, information may be loaded into computing device 18 from removable storage (e.g., a smart card, a flash drive, a removable disk) that enables the user 22, and/or other users to customize the implementation of computing device 18. Other exemplary input devices and techniques adapted for use with computing device 18 include, but are not limited to, an RS-232 port, RF link, an IR link, a modem (telephone, cable, etc.) and/or other devices. In some embodiments, the interactive visualization and/or other visualizations may be presented user 22 and/or other users via user interface 20.

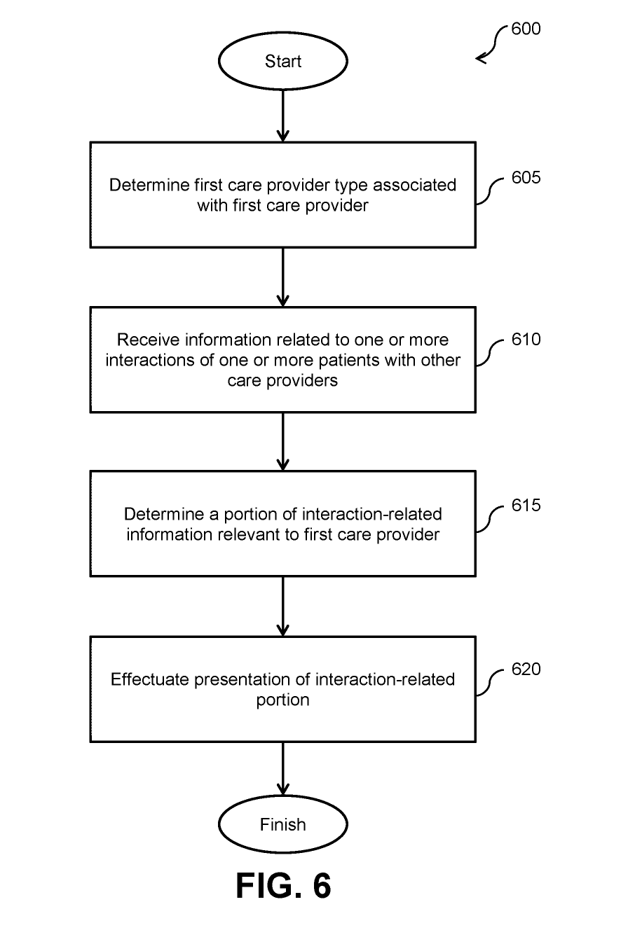

FIG. 6 illustrates an exemplary method 600 for facilitating visualization of interactions in a network of care providers with a system. The system comprises one or more processors and/or other components. The one or more processors are configured by machine readable instructions to execute computer program components. The computer program components comprise a provider type determination component, a communications component, a relevance determination component, a presentation component, and/or other components. The operations of method 600 presented below are intended to be illustrative. In some embodiments, method 600 may be accomplished with one or more additional operations not described, and/or without one or more of the operations discussed. Additionally, the order in which the operations of method 600 are illustrated in FIG. 6 and described below is not intended to be limiting.

In some embodiments, method 600 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 600 in response to instructions stored electronically on an electronic storage medium. The one or more processing devices may include one or more devices configured through hardware, firmware, and/or software to be specifically designed for execution of one or more of the operations of method 200.

At an operation 605, a first care provider type associated with a first care provider is determined. In some embodiments, the first care provider includes formal care providers (e.g., cardiologist, diabetic specialist nurse, health coach, home nurse), informal care providers (e.g., family member), and/or other care providers. In some embodiments, one or more parameters relevant to the first care provider type are determined. In some embodiments, the one or more parameters include one or more keywords, conditions, illnesses, diagnosis, medication, or interaction types relevant to the first care provider type. In some embodiments, operation 605 is performed by a processor component the same as or similar to provider type determination component 26 (shown in FIG. 1 and described herein).

At an operation 610, information related to one or more interactions of one or more patients with other care providers is received. In some embodiments, the interaction-related information includes information related to one or more interactions of a first patient with at least one of the other care providers. In some embodiments, the one or more interactions of the first patient may not be originating from the first care provider. In some embodiments, the one or more interactions of the first patient may include interactions with the first care provider. In some embodiments, operation 610 is performed by a processor component the same as or similar to communications component 28 (shown in FIG. 1 and described herein).

At an operation 615, at least a portion of the interaction-related information relevant to the first care provider is determined. In some embodiments, the interaction-related information portion being determined to be relevant to the first care provider is based on the one or more interactions of the first patient being relevant to the first care provider type. In some embodiments, the interaction-related information relevant to the first care provider is determined despite the one or more interactions of the first patient not originating from the first care provider. In some embodiments, operation 615 is performed by a processor component the same as or similar to relevance determination component 30 (shown in FIG. 1 and described herein).

At an operation 620, presentation of the interaction-related information portion is effectuated. In some embodiments, the presentation of the interaction-related information portion reflects one or more aspects of the one or more interactions of the first patient not originating from the first care provider. In some embodiments, operation 620 is performed by a processor component the same as or similar to presentation component 32 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system configured for visualization of interactions in a network of care providers, the system comprising one or more processors configured by machine-readable instructions to:
determine a first care provider type associated with a first care provider;
receive interaction-related information related to one or more interactions of one or more subjects with one or more other care providers, the interaction-related information including at least information related to one or more interactions of a first subject with at least one of the one or more other care providers, wherein the first care provider is not one of the one or more other care providers;
determine at least a portion of the interaction-related information relevant to the first care provider, the interaction-related information portion being determined to be relevant to the first care provider based on the one or more interactions of the first subject being relevant to the first care provider type; and
effectuate presentation of the interaction-related information portion such that the presentation of the interaction-related information portion reflects one or more aspects of the one or more interactions of the first subject originating from the one or more other care providers.

2. The system of claim 1, wherein the one or more processors are configured to:
determine one or more parameters relevant to the first care provider type, the one or more parameters including one or more keywords, conditions, illnesses, diagnosis, medication, or interaction types relevant to the first care provider type; and
determine the interaction-related information portion as being relevant to the first care provider based on the one or more interactions of the first subject matching the one or more parameters relevant to the first care provider type.

3. The system of claim 2, wherein the one or more processors are further configured to notify the first care provider with regard to a failed appointment by:
identifying whether the failed appointment is due to an appointment not being scheduled in time, a scheduled appointment not taking place, or the appointment being rescheduled;
determining whether appointment topic content of the failed appointment is relevant to the first care provider based on the appointment topic content matching the one or more parameters relevant to the first care provider type; and
responsive to a determination that the appointment topic content is relevant to the first care provider, notifying the first care provider with regard to the failed appointment.

4. The system of claim 1, wherein the received interaction-related information comprises one or more of a time, a place, a duration, a frequency, an intensity, the care provider type, discussed diagnosis, treatment steps, lab orders, forwarding to the one or more other care providers, medication subscriptions, self-reported compliance to therapy, or experienced symptoms and side effects corresponding to the one or more interactions of the one or more subjects.

5. The system of claim 1, wherein effectuating the presentation of the interaction-related portion comprises:
effectuating presentation of the one or more other care providers;
sorting the presentation of the one or more other care providers based on a chronological order of the first subject's one or more interactions with the one or more other care providers;
effectuating presentation of one or more interactions between the one or more other care providers; and
distinguishing between intensities of interactions between the first subject and the one or more other care providers by varying one or more of a thickness, a size, or a color of an outline corresponding to individual ones of the one or more other care providers.

6. The system of claim 1, wherein effectuating the presentation of the interaction-related information portion comprises:
effectuating presentation of one or more healthcare entities;
distinguishing between individual frequencies of interactions between the first subject with the one or more healthcare entities by varying one or more of a thickness, length, or color of one or more lines between the first subject and the one or more healthcare entities;
highlighting the most recent interaction between the first subject and the one or more healthcare entities; and
effectuating presentation of the first subject's interaction with the one or more other care providers at an individual one of the one or more healthcare entities responsive to a selection of the one or more healthcare entities.

7. A method for visualization of interactions in a network of care providers, the method being implement by a computer system including one or more processors configured by machine-readable instructions, the method comprising:
determining a first care provider type associated with a first care provider;
receiving interaction-related information related to one or more interactions of one or more subjects with one or more other care providers, the interaction-related information including at least information related to one or more interactions of a first subject with at least one of the one or more other care providers, wherein the first care provider is not one of the one or more other care providers;
determining at least a portion of the interaction-related information relevant to the first care provider, the interaction-related information portion being determined to be relevant to the first care provider based on the one or more interactions of the first subject being relevant to the first care provider type; and
effectuating presentation of the interaction-related information portion such that the presentation of the interaction-related information portion reflects one or more aspects of the one or more interactions of the first subject originating from the one or more other care providers.

8. The method of claim 7, further comprising:
determining one or more parameters relevant to the first care provider type, the one or more parameters including one or more keywords, conditions, illnesses, diagnosis, medication, or interaction types relevant to the first care provider type; and
determining, the interaction-related information portion as being relevant to the first care provider based on the one or more interactions of the first subject matching the one or more parameters relevant to the first care provider type.

9. The method of claim 8, further comprising notifying the first care provider with regard to a failed appointment by:
identifying whether the failed appointment is due to an appointment not being scheduled in time, a scheduled appointment not taking place, or the appointment being rescheduled;
determining whether appointment topic content of the failed appointment is relevant to the first care provider based on the appointment topic content matching the one or more parameters relevant to the first care provider type; and
responsive to a determination that the appointment topic content is relevant to the first care provider, notifying the first care provider with regard to the failed appointment.

10. The method of claim 7, wherein the received interaction-related information comprises one or more of a time, a place, a duration, a frequency, an intensity, the care provider type, discussed diagnosis, treatment steps, lab orders, forwarding to the one or more other care providers, medication subscriptions, self-reported compliance to therapy, or experienced symptoms and side effects corresponding to the one or more interactions of the one or more subjects.

11. The method of claim 7, wherein effectuating the presentation of the interaction-related portion comprises:
effectuating presentation of the one or more other care providers;
sorting the presentation of the one or more other care providers based on a chronological order of the first subject's one or more interactions with the one or more other care providers;
effectuating presentation of one or more interactions between the one or more other care providers; and
distinguishing between intensities of interactions between the first subject and the one or more other care providers by varying one or more of a thickness, a size, or a color of an outline corresponding to individual ones of the one or more other care providers.

12. The method of claim 7, wherein effectuating the presentation of the interaction-related information portion comprises:
effectuating presentation of one or more healthcare entities;
distinguishing between individual frequencies of interactions between the first subject with the one or more healthcare entities by varying one or more of a thickness, length, or color of one or more lines between the first subject and the one or more healthcare entities;
highlighting the most recent interaction between the first subject and the one or more healthcare entities; and
effectuating presentation of the first subject's interaction with the one or more other care providers at an individual one of the one or more healthcare entities responsive to a selection of the one or more healthcare entities.

13. A system for visualization of interactions in a network of care providers, the system comprising:
means for determining a first care provider type associated with a first care provider;
means for receiving interaction-related information related to one or more interactions of one or more subjects with one or more other care providers, the interaction-related information including information related at least to one or more interactions of a first subject with at least one of the one or more other care providers, wherein the first care provider is not one of the one or more other care providers;
means for determining at least a portion of the interaction-related information relevant to the first care provider, the interaction-related information portion being determined to be relevant to the first care provider based on the one or more interactions of the first subject being relevant to the first care provider type; and
means for effectuating presentation of the interaction-related information portion such that the presentation of the interaction-related information portion reflects one or more aspects of the one or more interactions of the first subject originating from the one or more other care providers.

14. The system of claim 13, further comprising:
means for determining one or more parameters relevant to the first care provider type, the one or more parameters including one or more keywords, conditions, illnesses, diagnosis, medication, or interaction types relevant to the first care provider type; and
means for determining the interaction-related information portion as being relevant to the first care provider based on the one or more interactions of the first subject matching the one or more parameters relevant to the first care provider type.

15. The system of claim 14, further comprising means for notifying the first care provider with regard to a failed appointment by:
means for identifying whether the failed appointment is due to an appointment not being scheduled in time, a scheduled appointment not taking place, or the appointment being rescheduled;
means for determining whether appointment topic content of the failed appointment is relevant to the first care provider based on the appointment topic content matching the one or more parameters relevant to the first care provider type; and
responsive to a determination that the appointment topic content is relevant to the first care provider, means for notifying the first care provider with regard to the failed appointment.

16. The system of claim 13, wherein the received interaction-related information comprises one or more of a time, a place, a duration, a frequency, an intensity, the care provider type, discussed diagnosis, treatment steps, lab orders, forwarding to the one or more other care providers, medication subscriptions, self-reported compliance to therapy, or experienced symptoms and side effects corresponding to the one or more interactions of the one or more subjects.

17. The system of claim 13, wherein the means for effectuating the presentation of the interaction-related portion comprises:
means for effectuating presentation of the one or more other care providers;
means for sorting the presentation of the one or more other care providers based on a chronological order of the first subject's one or more interactions with the one or more other care providers;
means for effectuating presentation of one or more interactions between the one or more other care providers; and
means for distinguishing between intensities of interactions between the first subject and the one or more other care providers by varying one or more of a thickness, a size, or a color of an outline corresponding to individual ones of the one or more other care providers.

18. The system of claim 13, wherein the means for effectuating the presentation of the interaction-related information portion comprises:
means for effectuating presentation of one or more healthcare entities;
means for distinguishing between individual frequencies of interactions between the first subject with the one or more healthcare entities by varying one or more of a thickness, length, or color of one or more lines between the first subject and the one or more healthcare entities;
means for highlighting the most recent interaction between the first subject and the one or more healthcare entities; and
means for effectuating presentation of the first subject's interaction with the one or more other care providers at an individual one of the one or more healthcare entities responsive to a selection of the one or more healthcare entities.
